(19) United States
(12) Patent Application Publication
(10) Pub. No.: US 2026/0275412 A1
(43) Pub. Date: Sep. 17, 2026

(54) A GENE SIGNATURE FOR DIAGNOSING STIMULATOR OF INTERFERON GENES (STING)-ASSOCIATED VASCULOPATHY WITH ONSET IN INFANCY (SAVI)

(71) Applicants: INSTITUT NATIONAL DE LA SANTÉ ET DE LA RECHERCHE MÉDICALE, Paris (FR); UNIVERSITÉ PARIS CITÉ, Paris (FR); ASSISTANCE PUBLIQUE-HÔPITAUX DE PARIS (APHP), Paris (FR); FONDATION IMAGINE, Paris (FR); SANOFI, Paris (FR)

(72) Inventors: Mickaël MENAGER, Paris (FR); Galina BOLDINA, Paris (FR); Camille CHALLEND DE CEVINS, Paris (FR); Frédéric RIEUX-LAUCAT, Paris (FR); Michel DIDIER, Paris (FR)

(21) Appl. No.: 19/168,610

(22) PCT Filed: Mar. 27, 2023
(86) PCT No.: PCT/IB2023/000159

§ 371 Date: Sep. 24, 2025

(51) Int. Cl. C12Q 1/6869; C12Q 1/6809; G16B 25/10; G16B 40/20; G16H 10/40; G16H 10/60; G16H 20/10

(52) U.S. Cl. CPC C12Q 1/6869; C12Q 1/6809; G16B 25/10; G16B 40/20; G16H 10/40; G16H 10/60; G16H 20/10; C12Q 2600/106; C12Q 2600/112; C12Q 2600/158

(57) ABSTRACT

Gain-of-function mutations in STING1, that codes for the Stimulator of Interferon Gene (STING), result in a severe autoinflammatory disease termed STING-associated vasculopathy with onset in infancy (SAVI). Although elevated type I interferon (IFN) production is thought to be the leading cause of the symptoms observed in patients, STING can induce a set of pathways, and their role in the onset and severity of SAVI remains to be elucidated. To address this point, the inventors compared a single-cell RNA sequencing (scRNA-seq) dataset of peripheral blood mononuclear cells (PBMCs) from SAVI patients to a dataset of healthy PBMCs treated with recombinant IFN-β. In particular, scRNA-seq clustering identified a patient-specific subset of monocytes, highly inflammatory and expressing a strong integrated stress response (ISR). Even more particularly, the inventors have identified at the transcriptomic level a STING-associated signature of 21 genes that is driven by STING activation, independently of type I IFN. This signature is therefore suitable to better distinguish SAVI from other type I interferonopathy.

### FIELD OF THE INVENTION

The present invention is in the field of medicine, in particular immunology.

### BACKGROUND OF THE INVENTION

Stimulator of Interferon genes (STING)-associated vasculopathy with onset in infancy (SAVI) is a rare monogenic disease characterized by uncontrolled production of type I interferons (IFNs)^{1,2}. Type I IFNs are the first line of defense against pathogenic infection. They lead to a robust activation of the immune system and increase the effector functions of immune cells while triggering apoptosis of infected cells. However, type I IFNs are highly potent cytokines that can become detrimental if uncontrolled. SAVI is a type I interferonopathy that appears during the first few weeks or months of life and is characterized by severe pulmonary and cutaneous manifestations³. It is caused by gain-of-function mutations in STING1, which codes for STING, a key component of innate immunity, capable of eliciting a potent innate immune response. About 70 cases of SAVI have been described in the literature⁴.

The current diagnosis of type I interferonopathies, including SAVI, relies on clinical evaluation followed by measurement of the level of expression of 6 interferon-stimulated genes in PBMCs of suspected patients. This approach is common to all type I interferonopathies, and subsequent exome sequencing (an expensive approach) is required to pinpoint a specific gene¹. There is therefore an unmet need for an easy-to-use, differential diagnosis strategy between the different type I interferonopathies.

In normal conditions, the STING pathway acts as a sensor of cytoplasmic nucleic acids, which are interpreted as a danger signal. Upon sensing of cytoplasmic nucleic acids, STING is activated and changes conformation to traffic from the endoplasmic reticulum (ER) to the Golgi apparatus^{5,6}. There, STING phosphorylates IRF3 and NF-κB, leading to the expression of type I IFNs and other proinflammatory cytokines⁷⁻⁹. Other molecular pathways can also be triggered by STING, such as ER stress and senescence^{10,11}. Moreover, STING can induce cell type-specific programs, such as apoptosis, which is induced in T cells but not in macrophages or dendritic cells¹².

In SAVI patients, STING is constitutively activated and present on the membrane of the Golgi apparatus^{1,13}. SAVI is associated with T cell lymphopenia, and an imbalance between memory and naïve CD8⁺ T cells, with decreased percentages of effector and memory T cells^{3,14}. A low number of circulating NK cells has also been reported in some patients¹⁵. At the molecular levels, IFN-α/β and IFN-stimulated genes (ISGs) are consistently upregulated in all patients¹⁻³. Patients have elevated inflammatory cytokines plasma concentrations, including Tumor Necrosis Factor-α (TNF-α) and Interleukin-6 (IL-6)¹⁶, suggesting the involvement of the NF-κB pathway. Patients also have increase expression of genes involved in apoptosis, cell adhesion, and coagulation². Many pathways are thought to be triggered by STING activation. Some of them, such as type I IFNs production and NF-κB activation, are upregulated in SAVI patients. Others, such as T cell activation or increased ER stress, have been demonstrated in murine models but have yet to be confirmed in SAVI patients¹¹.

As uncontrolled production of type I IFNs is a hallmark of SAVI, patients can be treated with Janus kinase (JAK)-inhibitors. They block the signal transduction upon IFN-α/β binding to their receptor. The first line of JAK-inhibitor treatment is Ruxolitinib or Baricitinib, both inhibitors of JAK1 and JAK2 which act downstream of the IFN-α/β receptor (IFNAR). Tofacitinib, another JAK-inhibitor targeting JAK1 and JAK3 has also been used. These treatments lead to partial improvements of the symptoms^{3,17,18}. However, in cases where the interstitial lung disease is too advanced, JAK-inhibitor treatment has proved inefficient^{3,17} suggesting a role of STING activation independent of type I IFN signaling in SAVI. A deep understanding of the pathways modulated in patients is crucial to effectively treat the disease.

### SUMMARY OF THE INVENTION

The present invention is defined by the claims. In particular, the present invention relates to a signature of 21 genes for diagnosing Stimulator of Interferon genes (STING)-associated vasculopathy with onset in infancy (SAVI).

### DETAILED DESCRIPTION OF THE INVENTION

Gain-of-function mutations in STING1, that codes for the Stimulator of Interferon Gene (STING), result in a severe autoinflammatory disease termed STING-associated vasculopathy with onset in infancy (SAVI). Although elevated type I interferon (IFN) production is thought to be the leading cause of the symptoms observed in patients, STING can induce a set of pathways, and their role in the onset and severity of SAVI remains to be elucidated. To address this point, the inventors compared a single-cell RNA sequencing (scRNA-seq) dataset of peripheral blood mononuclear cells (PBMCs) from SAVI patients to a dataset of healthy PBMCs treated with recombinant IFN-β. They revealed a loss of mucosal associated invariant T cells and CD56^{bright} natural killer cells in SAVI patients, not replicated in IFN-β-treated PBMC. Patient T cells are in an activated state associated with senescence and apoptosis, dependent on type I IFNs. Inferring cell to cell communication, from scRNA-seq predicted monocytes as potential drivers of this T cell phenotype and was supported by plasma cytokines measurement, with high CCL3, CCL4 and IL-6. Furthermore, scRNA-seq clustering identified a patient-specific subset of monocytes, highly inflammatory and expressing a strong integrated stress response (ISR). It also pinpointed to a patient with lower ISR, allowing the inventors to identify a secondary mutation in PERK, that was recently shown to be activated by STING to trigger the ISR. Finally, based on the identification of this patient-specific subset of monocytes and the exploration of IFN-β stimulated PBMCs from healthy donors, the inventors developed a strategy to propose a transcriptomic signature specific of STING activation and independent of type I IFN.

The first object of the present invention relates to a method for diagnosing a STING-associated vasculopathy with onset in infancy (SAVI) in a subject, comprising determining in a sample obtained from the subject the expression level of one or more gene(s) selected from the group consisting of AC253572.2, ADRB2, ANKRD28, ANXA1, AQP9, AZIN1-AS1, IL1B, JARID2, KLF4, MIR4435-2HG, OTUD1, PLAUR, PTGS2, PTX3, SELENOK, TGIF1, TREM1, TSC22D2, ZBTB43, ZFP36L2 and ZNF503 wherein the expression level indicates that the subject suffers or not from a SAVI.

As used herein, the term “subject” is intended for a human or non-human mammal. Typically, the subject is affected or likely to suffer from a SAVI. In some embodiments, the subject is a human infant. In some embodiments, the subject is a human child.

As used herein, the term “STING-associated vasculopathy with onset in infancy” or “SAVI” or has its general meaning in the art and refers to a rare, genetic autoinflammatory disorder, type I interferonopathy due to constitutive STING (STimulator of INterferon Genes) activation, characterized by neonatal or infantile onset systemic inflammation and small vessel vasculopathy resulting in severe skin, pulmonary and joint lesions. Patients present with intermittent low-grade fever, recurrent cough and failure to thrive, in association with progressive interstitial lung disease, polyarthritis and violaceous scaling lesions on fingers, toes, nose, cheeks, and ears (which are exacerbated by cold exposure) that often progress to chronic acral ulceration, necrosis and autoamputation.

As used herein term “diagnosing” refers to classifying a disease or a symptom, determining a severity of the disease, monitoring disease progression, forecasting an outcome of a disease and/or prospects of recovery. In the context of the invention, the method according to the invention allows to diagnose a SAVI.

Typically, high expression levels indicate that the subject suffers from a SAVI whereas low expression levels indicate that the subject do not suffer from a SAVI.

As used herein, the term “high” refers to a measure that is greater than normal, greater than a standard such as a predetermined reference value or a subgroup measure or that is relatively greater than another subgroup measure. For example, high levels of expression refer to a level of expression that is greater than a normal expression level. A normal expression level may be determined according to any method available to one skilled in the art. High level of expression may also refer to a level that is equal to or greater than a predetermined reference value, such as a predetermined cutoff. High level of expression may also refer to a level of expression wherein a high expression subgroup has relatively greater levels of expression than another subgroup. For example, without limitation, according to the present specification, two distinct patient subgroups can be created by dividing samples around a mathematically determined point, such as, without limitation, a median, thus creating a subgroup whose measure is high (i.e., higher than the median) and another subgroup whose measure is low. In some cases, a “high” level may comprise a range of level that is very high and a range of level that is “moderately high” where moderately high is a level that is greater than normal, but less than “very high”.

As used herein, the term “low” refers to a level that is less than normal, less than a standard such as a predetermined reference value or a subgroup measure that is relatively less than another subgroup level. For example, low level of expression means a level of expression that is less than a normal level of in a particular set of samples of subjects. A normal level of expression measure may be determined according to any method available to one skilled in the art. Low level of expression may also mean a level that is less than a predetermined reference value, such as a predetermined cutoff. Low level of expression may also mean a level wherein a low level expression subgroup is relatively lower than another subgroup. For example, without limitation, according to the present specification, two distinct subject subgroups can be created by dividing samples around a mathematically determined point, such as, without limitation, a median, thus creating a group whose measure is low (i.e., less than the median) with respect to another group whose measure is high (i.e., greater than the median).

As used herein, the term “predetermined reference value” refers to a threshold value or a cut-off value. A “threshold value”, “reference value” or “cut-off value” can be determined experimentally, empirically, or theoretically. A threshold value can also be arbitrarily selected based upon the existing experimental and/or clinical conditions, as would be recognized by a person of ordinary skilled in the art. For example, retrospective measurement of the level of the marker of the invention (e.g. expression) in properly banked historical subject samples may be used in establishing the predetermined corresponding reference value. In some embodiments, the predetermined corresponding reference value is the median measured in the population of the subjects for the marker of in the invention (expression for example). In some embodiments, the threshold value has to be determined in order to obtain the optimal sensitivity and specificity according to the function of the test and the benefit/risk balance (clinical consequences of false positive and false negative). Typically, the optimal sensitivity and specificity (and so the threshold value) can be determined using a Receiver Operating Characteristic (ROC) curve based on experimental data. For example, after determining the level of the marker of the invention (expression for example) in a group of reference, one can use algorithmic analysis for the statistic treatment of the levels determined in samples to be tested, and thus obtain a classification standard having significance for sample classification. The full name of ROC curve is receiver operator characteristic curve, which is also known as receiver operation characteristic curve. It is mainly used for clinical biochemical diagnostic tests. ROC curve is a comprehensive indicator the reflects the continuous variables of true positive rate (sensitivity) and false positive rate (1-specificity). It reveals the relationship between sensitivity and specificity with the image composition method. A series of different cut-off values (thresholds or critical values, boundary values between normal and abnormal results of diagnostic test) are set as continuous variables to calculate a series of sensitivity and specificity values. Then sensitivity is used as the vertical coordinate and specificity is used as the horizontal coordinate to draw a curve. The higher the area under the curve (AUC), the higher the accuracy of diagnosis. On the ROC curve, the point closest to the far upper left of the coordinate diagram is a critical point having both high sensitivity and high specificity values. The AUC value of the ROC curve is between 1.0 and 0.5. When AUC>0.5, the diagnostic result gets better and better as AUC approaches 1. When AUC is between 0.5 and 0.7, the accuracy is low. When AUC is between 0.7 and 0.9, the accuracy is moderate. When AUC is higher than 0.9, the accuracy is quite high. This algorithmic method is preferably done with a computer. Existing software or systems in the art may be used for the drawing of the ROC curve, such as: MedCalc 9.2.0.1 medical statistical software, SPSS 9.0, ROCPOWER.SAS, DESIGNROC.FOR, MULTIREADER POWER.SAS, CREATE-ROC.SAS, GB STAT VI0.0 (Dynamic Microsystems, Inc. Silver Spring, Md., USA), etc.

A further object of the present invention relates to a method for discriminating a STING-associated vasculopathy with onset in infancy (SAVI) from another one type I interferonopathy in a subject comprising determining in a sample obtained from the subject the expression level of one or more gene(s) selected from the group consisting of AC253572.2, ADRB2, ANKRD28, ANXA1, AQP9, AZIN1-AS1, IL1B, JARID2, KLF4, MIR4435-2HG, OTUD1, PLAUR, PTGS2, PTX3, SELENOK, TGIF1, TREM1, TSC22D2, ZBTB43, ZFP36L2 and ZNF503 wherein the expression level indicates that the subject suffers or not from a SAVI.

As used herein, the term “type I interferonopathy” has its general meaning in the art and refers to a subgroup of autoinflammatory diseases caused by mutations in genes associated with proteasome degradation or cytoplasmic RNA- and DNA-sensing pathways. The term “interferonopathy” first appeared in 2003, when some authors identified phenotypic overlaps between Aicardi-Goutieres syndrome (AGS) encephalopathy, viral congenital infections, and some autoimmune diseases such as systemic lupus erythematosus (SLE), postulating a common pathological feature as an upregulation of interferon (IFN) a activity (Crow, Yanick J. “Type I interferonopathies: a novel set of inborn errors of immunity.” Annals of the New York Academy of Sciences 1238.1 (2011): 91-98). In particular, the term includes Aicardi-Goutières syndrome (AGS), STING-associated vasculopathy with onset in infancy (SAVI), and chronic atypical neutrophilic dermatosis with lipodystrophy and elevated temperature syndrome. Patients with type I interferonopathy shared several clinical characteristics, including bilateral calcifications of the basal ganglia, chilblain-like rashes, and liver dysfunction. Each subtype includes disease-specific severe complications, such as early-onset encephalopathy associated with AGS and pulmonary hypertension observed in patients diagnosed with STING-associated vasculopathy with onset in infancy.

As used herein, the term “discriminating” refers to identify, observe a difference or distinguish two groups.

Typically, high expression levels indicate that the subject suffers from a SAVI whereas low expression levels indicate that the subject suffers from another type I interferonopathy.

A further object of the present invention relates to a method of determining whether a subject suffering from a SAVI achieves a response to a therapy comprising determining the expression level of one or more gene(s) selected from the group consisting of AC253572.2, ADRB2, ANKRD28, ANXA1, AQP9, AZIN1-AS1, IL1B, JARID2, KLF4, MIR4435-2HG, OTUD1, PLAUR, PTGS2, PTX3, SELENOK, TGIF1, TREM1, TSC22D2, ZBTB43, ZFP36L2 and ZNF503 wherein said level indicates whether the subject achieves or not a response to the therapy.

Typically, low levels of expression indicate that the subject achieves a response to the therapy whereas high levels of expression indicate that the subject does not achieve a response to the therapy. In some embodiments, the predetermined level is the level determined in a sample obtained from the subject before the treatment.

As used herein, the terms “achieve a response” or “respond” refer to the response to a therapy of the subject suffering from a SAVI. Typically such therapy induces, ameliorates or otherwise causes an improvement in the pathological symptoms, disease progression or physiological conditions associated with or resistance to succumbing to a SAVI. In particular, in the context of the invention, the term “respond” refers to the ability of the therapy to lead to an improvement of the pathological symptoms, thus, the subject presents a clinical improvement compared to the subject who does not receive the therapy. The subject is considered as a “responder” to the treatment. The term “not respond” refers to a subject who does not present any clinical improvement to the treatment with the therapy. This subject is considered as a “non-responder” to the therapy. Accordingly, the subject is considered as “non-responder” has a particular monitoring in the therapeutic regimen.

In some embodiments, the therapy consists in administering to the subject a therapeutically effective amount of JAK inhibitor.

As used herein the term “JAK” has its general meaning in the art and refers to the family of Janus kinases (JAKs) which are cytoplasmic tyrosine kinases that transduce cytokine signaling from membrane receptors to STAT transcription factors. Four JAK family members are described, JAK1, JAK2, JAK3 and TYK2 and the term JAK may refer to all the JAK family members collectively or one or more of the JAK family members as the context indicates. As used herein the term “JAK inhibitor” is intended to mean any compound that inhibits the activity or expression of at least one of the JAK-family member. JAK inhibitors down-regulate the quantity or activity of JAK molecules. JAK inhibitors are well known in the art and typically include AG490, AUB-6-96, AZ960, AZD1480, baricitinib (LY3009104, INCB28050), BMS-911543, CEP-701, CMP6, CP352,664, CP690,550, CYT-387, INCB20, Jak2-IA, lestaurtinib (CEP-701), LS104, LY2784544, NS018, pacritinib (SB1518), Pyridone 6, ruxolitinib (INCB018424), SB1518, TG101209, TG101348 (SAR302503), tofacitinib (CP-690,550), WHI-PI 54, WP1066, XL019, and XLOI 9.

A further object of the present invention relates to a method of determining whether a subject suffering from a type I interferonopathy is eligible for treatment with a STING antagonist comprising determining the expression level of one or more gene(s) selected from the group consisting of AC253572.2, ADRB2, ANKRD28, ANXA1, AQP9, AZIN1-AS1, IL1B, JARID2, KLF4, MIR4435-2HG, OTUD1, PLAUR, PTGS2, PTX3, SELENOK, TGIF1, TREM1, TSC22D2, ZBTB43, ZFP36L2 and ZNF503 wherein said level indicates whether the subject is eligible for treatment with a STING antagonist.

Typically, high levels of expression indicate that the subject is eligible for treatment with a STING antagonist, whereas low levels of expression indicate that the subject is not eligible for treatment with a STING antagonist.

As used herein, the term “STING” has its general meaning in the art and refers to the stimulator of interferon genes (STING) protein, also known as transmembrane protein 173 (TMEM173) and MPYS/MITA/ERIS is a protein that in humans is encoded by the STING1 gene. As used herein the term “STING antagonist” is intended to mean compounds inhibit the activity or expression of STING. Example of STING antagonists include those described in Guerini, Danilo. “STING agonists/antagonists: their potential as therapeutics and future developments.” Cells 11.7 (2022): 1159; Haag, Simone M., et al. “Targeting STING with covalent small-molecule inhibitors.” Nature 559.7713 (2018): 269-273.; Hong, Ze, et al. “STING inhibitors target the cyclic dinucleotide binding pocket.” Proceedings of the National Academy of Sciences 118.24 (2021): e2105465118.; Ding, Chunyong, et al. “Small molecules targeting the innate immune cGAS-STING-TBK1 signaling pathway.” Acta Pharmaceutica Sinica B 10.12 (2020): 2272-2298 and Decout, Alexiane, et al. “The cGAS-STING pathway as a therapeutic target in inflammatory diseases.” Nature Reviews Immunology 21.9 (2021): 548-569.

In some embodiments, the sample is a sample of monocytes. As used herein, the term “monocyte” has its general meaning in the art and refers to a large mononuclear phagocyte of the peripheral blood. The main phenotypic markers of human monocyte cells include CD11b, CD11c, CD33 and CD115. Methods for isolating monocytes are well known in the art and typically include on cell sorting methods such as fluorescence activated cell sorting (FACS) or magnetic activated cell sorting (MACS). For instance, non-monocytes cells may be magnetically labeled with a cocktail of monoclonal antibodies chosen antibodies directed against CD3, CD7, CD19, CD56, CD123 and CD235a. Kits for isolation of monocytes are commercially available from Miltenyi Biotec (Auburn, CA, USA), Stem Cells Technologies (Vancouver, Canada) or Dynal Bioech (Oslo, Norway).

In some embodiments, the sample also comprises dendritic cells. As used herein, the term “Dendritic cells” or “DCs” refer to any member of a diverse population of morphologically similar cell types found in lymphoid or non-lymphoid tissues. These cells are characterized by their distinctive morphology, high levels of surface MHC-class II expression (Steinman, et al., Ann. Rev. Immunol. 9:271 (1991); incorporated herein by reference for its description of such cells).

In the present specification, the name of each of the gene of interest refers to the internationally recognized name of the corresponding gene, as found in internationally recognized gene sequences and protein sequences databases, including in the database from the HUGO Gene Nomenclature Committee. In the present specification, the name of each of the various immune markers of interest may also refer to the internationally recognized name of the corresponding gene, as found in the internationally recognized gene sequences and protein sequences database Genbank. Through these internationally recognized sequence databases, the nucleic acid and the amino acid sequences corresponding to each of the immune marker of interest described herein may be retrieved by the one skilled in the art.

Methods for determining the expression level of a gene product such as nucleic acid (e.g. RNA) are also well known in the art. Conventional methods typically involve polymerase chain reaction (PCR). For instance, U.S. Pat. Nos. 4,683,202, 4,683,195, 4,800,159, and 4,965,188 disclose conventional PCR techniques. PCR typically employs two oligonucleotide primers that bind to a selected target nucleic acid sequence. Primers useful in the present invention include oligonucleotides capable of acting as a point of initiation of nucleic acid synthesis within the target nucleic acid sequence. A primer can be purified from a restriction digest by conventional methods, or it can be produced synthetically. PCR involves use of a thermostable polymerase. Typically, the polymerase is a Taq polymerase (i.e. Thermus aquaticus polymerase). Quantitative PCR is typically carried out in a thermal cycler with the capacity to illuminate each sample with a beam of light of a specified wavelength and detect the fluorescence emitted by the excited fluorophore. The thermal cycler is also able to rapidly heat and chill samples, thereby taking advantage of the physicochemical properties of the nucleic acids and thermal polymerase. In order to detect and measure the amount of amplicon (i.e. amplified target nucleic acid sequence) in the sample, a measurable signal has to be generated, which is proportional to the amount of amplified product. All current detection systems use fluorescent technologies. Some of them are non-specific techniques, and consequently only allow the detection of one target at a time. Alternatively, specific detection chemistries can distinguish between non-specific amplification and target amplification. These specific techniques can be used to multiplex the assay, i.e. detecting several different targets in the same assay. For example, SYBR® Green I probes, High Resolution Melting probes, TaqMan® probes, LNA® probes and Molecular Beacon probes can be suitable. TaqMan® probes are the most widely used type of probes. They were developed by Roche (Basel, Switzerland) and ABI (Foster City, USA) from an assay that originally used a radio-labelled probe (Holland et al. 1991), which consisted of a single-stranded probe sequence that was complementary to one of the strands of the amplicon. A fluorophore is attached to the 5′ end of the probe and a quencher to the 3′ end. The fluorophore is excited by the machine and passes its energy, via FRET (Fluorescence Resonance Energy Transfer) to the quencher. Traditionally, the FRET pair has been conjugated to FAM as the fluorophore and TAMRA as the quencher. In a well-designed probe, FAM does not fluoresce as it passes its energy onto TAMRA. As TAMRA fluorescence is detected at a different wavelength to FAM, the background level of FAM is low. The probe binds to the amplicon during each annealing step of the PCR. When the Taq polymerase extends from the primer which is bound to the amplicon, it displaces the 5′ end of the probe, which is then degraded by the 5′-3′ exonuclease activity of the Taq polymerase. Cleavage continues until the remaining probe melts off the amplicon. This process releases the fluorophore and quencher into solution, spatially separating them (compared to when they were held together by the probe). This leads to an irreversible increase in fluorescence from the FAM and a decrease in the TAMRA.

In some embodiments, the expression level of the gene(s) is determined by RNA sequencing, and more particularly by single cell RNA sequencing.

As used, the term “RNA sequencing” refers to sequencing performed on RNA (or cDNA) instead of DNA, where typically, the primary goal is to measure expression levels, detect fusion transcripts, alternative splicing, and other genomic alterations that can be better assessed from RNA. RNA sequencing typically includes whole transcriptome sequencing or targeted exome sequencing. In some embodiments, targeted exome sequencing may be preferred. As used herein, the term “whole transcriptome sequencing” refers to the use of high throughput sequencing technologies to sequence the entire transcriptome in order to get information about a sample's RNA content. As used herein, the term “targeted exome sequencing” refers to the use of high throughput sequencing technologies to sequence some specific targeted sequencing. RNA sequencing can be done with a variety of platforms for example, the Genome Analyzer (Illumina, Inc., San Diego, Calif.) and the SOLiD™ Sequencing System (Life Technologies, Carlsbad, Calif.), However, any platform useful for whole transcriptome sequencing may be used. Typically, the RNA is extracted, and ribosomal RNA may be deleted as described in U.S. Pub, No. 2011/0111409. cDNA sequencing libraries may be prepared that are directional and single or paired-end using commercially available kits such as the ScriptSeq™ M mRNA-Seq Library Preparation Kit (Epicenter Biotechnologies, Madison, Wis.). The libraries may also be barcoded for multiplex sequencing using commercially available barcode primers such as the RNA sequencing Barcode Primers from Epicenter Biotechnologies (Madison, Wis.). PCR is then carried out to generate the second strand of cDNA to incorporate the barcodes and to amplify the libraries. After the libraries are quantified, the sequencing libraries may be sequenced. Nucleic acid sequencing technologies are suitable methods for expression analysis. The principle underlying these methods is that the number of times a cDNA sequence is detected in a sample is directly related to the relative RNA levels corresponding to that sequence. These methods are sometimes referred to by the term Digital Gene Expression (DOE) to reflect the discrete numeric property of the resulting data. Early methods applying this principle were Serial Analysis of Gene Expression (SAGE) and Massively Parallel Signature Sequencing (MPSS). See, e.g., S. Brenner, et al., Nature Biotechnology 18(6):630-634 (2000). Typically RNA sequencing uses Next Generation Sequencing or NGS. As used herein, the term “Next Generation Sequencing” (NGS) refers to a relatively new sequencing technique as compared to the traditional Sanger sequencing technique. For review, see Shendure et al., Nature Biotech., 26(10): 1135-45 (2008), which is hereby incorporated by reference into this disclosure. For purpose of this disclosure, NGS may include cyclic array sequencing, microelectrophoretic sequencing, sequencing by hybridization, among others. By way of example, in a typical NGS using cyclic-array methods, genomic DNA or cDNA library is first prepared, and common adaptors may then be ligated to the fragmented genomic DNA or cDNA. Different protocols may be used to generate jumping libraries of mate-paired tags with controllable distance distribution. An array of millions of spatially immobilized PCR colonies or “polonies” is generated with each polonies consisting of many copies of a single shotgun library fragment. Because the polonies are tethered to a planar array, a single microliter-scale reagent volume can be applied to manipulate the array features in parallel, for example, for primer hybridization or for enzymatic extension reactions. Imaging-based detection of fluorescent labels incorporated with each extension may be used to acquire sequencing data on all features in parallel. Successive iterations of enzymatic interrogation and imaging may also be used to build up a contiguous sequencing read for each array feature. As used herein, the term “single-cell RNA sequencing” has its general meaning in the art and examines the sequence information from individual cells with optimized next-generation sequencing technologies. The method provides a higher resolution of cellular differences and a better understanding of the function of an individual cell in the context of its microenvironment (Eberwine, James, et al. “The promise of single-cell sequencing.” Nature methods 11.1 (2014): 25-27).

In some embodiments, a score that is a composite of the levels of expression of the different genes included in the signature is calculated and compared to a predetermined reference value for determining whether:
the subject suffers from a SAVI,
the subject achieves a response to treatment and/or
the subject is eligible for a treatment with a STING antagonist.

As used herein, the term “score” refers to a numeric value that is derived by combining one or more parameter in a mathematic algorithm or formula. Combining the parameters can be accomplished for example by multiplying each expression level with a defined and specified coefficient and summing up such products to yield a score.

In some embodiments, the method of the present invention involves use of an algorithm.

In some embodiments, the method of the present invention comprises the steps of:
a) determining the expression level of at least one gene selected from the group consisting of AC253572.2, ADRB2, ANKRD28, ANXA1, AQP9, AZIN1-AS1, IL1B, JARID2, KLF4, MIR4435-2HG, OTUD1, PLAUR, PTGS2, PTX3, SELENOK, TGIF1, TREM1, TSC22D2, ZBTB43, ZFP36L2 and ZNF503,
b) implementing an algorithm on data comprising or consisting of the expression levels determined at step a), as to obtain an algorithm output, the implementing step being computer-implemented; and
c) determining whether:
the subject suffers from a SAVI,
the subject achieves a response to treatment and/or
the subject is eligible for a treatment with a STING antagonist.

As used herein, the term “algorithm” is any mathematical equation, algorithmic, analytical or programmed process, or statistical technique that takes one or more continuous parameters and calculates an output value, sometimes referred to as an “index” or “index value”.

Non-limiting examples of algorithms include sums, ratios, and regression operators, such as coefficients or exponents, biomarker value transformations and normalizations (including, without limitation, those normalization schemes based on clinical parameters, such as gender, age, or ethnicity), rules and guidelines, statistical classification models, and neural networks trained on historical populations. Non-limiting examples of algorithms thus include logistic regression, linear regression, random forests, classification and regression trees (C&RT), boosted trees, neural networks (NN), artificial neural networks (ANN), neuro fuzzy networks (NFN), network structures, perceptrons such as multi-layer perceptrons, multi-layer feed-forward networks, support vector machines (e.g., Kernel methods), multivariate adaptive regression splines (MARS), Levenberg-Marquardt algorithms, Gauss-Newton algorithms, mixtures of Gaussians, gradient descent algorithms, learning vector quantization (LVQ), and combinations thereof. Of particular use in combining parameters are linear and non-linear equations and statistical classification analyses to determine the relationship between levels of said parameters and the objective response to the preoperative adjuvant therapy. Of particular interest are structural and syntactic statistical classification algorithms, and methods of risk index construction, utilizing pattern recognition features, including established techniques such as cross-correlation, Principal Components Analysis (PCA), factor rotation, Logistic Regression (LogReg), Linear Discriminant Analysis (LDA), Eigengene Linear Discriminant Analysis (ELDA), Support Vector Machines (SVM), Random Forest (RF), Recursive Partitioning Tree (RPART), as well as other related decision tree classification techniques, Shrunken Centroids (SC), StepAIC, Kth-Nearest Neighbor, Boosting, Decision Trees, Neural Networks, Bayesian Networks, Support Vector Machines, and Hidden Markov Models, among others. Other techniques may be used in survival and time to event hazard analysis, including Cox, Weibull, Kaplan-Meier and Greenwood models well known to those of skill in the art.

In some embodiments, the method of the present invention comprises the use of a machine learning algorithm. The machine learning algorithm may comprise a supervised learning algorithm. Examples of supervised learning algorithms may include Average One-Dependence Estimators (AODE), Artificial neural network (e.g., Backpropagation), Bayesian statistics (e.g., Naive Bayes classifier, Bayesian network, Bayesian knowledge base), Case-based reasoning, Decision trees, Inductive logic programming, Gaussian process regression, Group method of data handling (GMDH), Learning Automata, Learning Vector Quantization, Minimum message length (decision trees, decision graphs, etc.), Lazy learning, Instance-based learning Nearest Neighbor Algorithm, Analogical modeling, Probably approximately correct learning (PAC) learning, Ripple down rules, a knowledge acquisition methodology, Symbolic machine learning algorithms, Subsymbolic machine learning algorithms, Support vector machines, Random Forests, Ensembles of classifiers, Bootstrap aggregating (bagging), and Boosting. Supervised learning may comprise ordinal classification such as regression analysis and Information fuzzy networks (IFN). Alternatively, supervised learning methods may comprise statistical classification, such as AODE, Linear classifiers (e.g., Fisher's linear discriminant, Logistic regression, Naive Bayes classifier, Perceptron, and Support vector machine), quadratic classifiers, k-nearest neighbor, Boosting, Decision trees (e.g., C4.5, Random forests), Bayesian networks, and Hidden Markov models. The machine learning algorithms may also comprise an unsupervised learning algorithm. Examples of unsupervised learning algorithms may include artificial neural network, Data clustering, Expectation-maximization algorithm, Self-organizing map, Radial basis function network, Vector Quantization, Generative topographic map, Information bottleneck method, and IBSEAD. Unsupervised learning may also comprise association rule learning algorithms such as Apriori algorithm, Eclat algorithm and FP-growth algorithm. Hierarchical clustering, such as Single-linkage clustering and Conceptual clustering, may also be used. Alternatively, unsupervised learning may comprise partitional clustering such as K-means algorithm and Fuzzy clustering. In some embodiments, the machine learning algorithms comprise a reinforcement learning algorithm Examples of reinforcement learning algorithms include, but are not limited to, temporal difference learning, Q-learning and Learning Automata. Alternatively, the machine learning algorithm may comprise Data Pre-processing.

In some embodiments, the algorithm is implemented on a computer using well-known computer processors, memory units, storage devices, computer software, and other components. Typically, the computer contains a processor, which controls the overall operation of the computer by executing computer program instructions which define such operation. The computer program instructions may be stored in a storage device (e.g., magnetic disk) and loaded into memory when execution of the computer program instructions is desired. The computer also includes other input/output devices that enable user interaction with the computer (e.g., display, keyboard, mouse, speakers, buttons, etc.). One skilled in the art will recognize that an implementation of an actual computer could contain other components as well.

The invention will be further illustrated by the following FIGURES and examples. However, these examples and FIGURES should not be interpreted in any way as limiting the scope of the present invention.

### EXAMPLE

### Methods:

### Sample Collection of the SAVI Dataset

Peripheral blood samples were collected on lithium heparin. Blood samples were centrifuged at 2,300 rpm (1,065 g) for 10 minutes to collect supernatant (plasma), which was stored at −20° C. Peripheral blood mononuclear cells (PBMCs) were isolated by density gradient centrifugation (2,200 rpm or 974 g without break for 30 minutes) using Ficoll (Eurobio Scientific, Les Ulis, France). After centrifugation, the pellet was resuspended in Phosphate-Buffered Saline (PBS) (Thermo Fisher scientific, Illkirch, France) and cells were centrifuged at 1500 rpm (453 g) for 5 minutes. Finally, the pellet was frozen in a medium containing 90% of Fetal Bovine Serum (FBS) (GIBCO, Thermo Fisher scientific, Illkirch, France) and 10% of Dimethyl Sulfoxide (DMSO) (Sigma Aldrich, St. Quentin Fallavier, France), and stored in liquid nitrogen.

### Cell Phenotyping by Flow Cytometry

For each sample of the SAVI dataset, 500,000 cells were stained. After an initial wash and 350 g centrifugation, Zombie NIR™ Fixable Viability kit was used to stain dead cells according to manufacturer instructions. Cells were washed and resuspended in 200 μL of wash buffer (PBS, 5% BSA, 1 mM EDTA), before staining with 90 μL of antibody mix with antibodies diluted 1/50. Fc Block (Biolegend Europ, Netherland) and wash buffer were used to avoid unspecific bindings. For each panel, extracellular staining has been performed for 30 min at 4° C. protected from light. Samples were analyzed using the Sony SP6800 spectral flow cytometers.

### IFN-β Stimulation of Healthy PBMC

Buffy coats from three healthy young male (19 to 22 years old) were obtained from the Etablissement Français du Sang. PBMCs were isolated using Ficoll (Eurobio Scientific, Les Ulis, France) density gradient and centrifugation at 800 g without brakes for 25 minutes. Following centrifugation, cells were washed in wash buffer and centrifuged at 160 g for 9 minutes at 4° C. and the pellet was resuspended in 10 mL of wash buffer. Cells were plated in 96-wells tissue culture plates, with 150,000 cells per wells in an initial volume of 125 μL. One plate was made for each donor for each timepoint to plate a total of 15 million cells per condition.

Cells were challenged with 25 μL of recombinant IFN-β (Human interferon beta 1a, mammalian, Catalog No 11410-2, PBL assay Science, USA) at 6,000 IU/mL for a final concentration of 1000 U/mL per well, at one of several timepoints: 1 h, 2 h, 4 h, 8 h, 12 h, 24 h or 36 h. Stimulations were done so that all cells, even unstimulated, would be kept a total of 36 h in culture. After 36 h of culture at 37° C., 5% of CO2, cells were collected and counted. 10,000 cells were sent to scRNA-seq and 2 million to cell phenotyping by CyTOF. Supernatant was frozen at −20° C. and kept for analysis of secreted proteins.

### Cell Phenotyping by Cytometry by Time of Flight

High dimensional immune profiling of the cultured cells was obtained using the Maxpar Direct Immune Profiling System (Fluidigm, Inc France) with a 30-marker antibody panel, for CyTOF (Cytometry by Time Of Flight). Briefly, 2 million cells were washed with MaxPar Cell Staining Buffer, and the cells were transferred in polypropylene tubes and incubated at room temperature for 10 minutes with 5 μL of TruStain FcX (Biolegend Europ, Netherland). Cells were then transferred into the tube containing the dry antibody cocktail, vortexed and incubated for 30 minutes at room temperature. After washing, the cells were fixed with 1 mL of 1.6% paraformaldehyde solution (SigmaAldrich, France) and incubated at room temperature for 10 minutes. After washing, 1 mL CellID intercalator-Ir at 125 nM (pentamethylcyclopentadienyl-Ir (III)-dipyridophenazine, Fluidigm, Inc France) was added onto the cells and incubated overnight. Cells were then stored at −80° C. until acquisition.

Cells were thawed at room temperature then washed 3 times in Maxpar Cell Acquisition Solution Plus (CAS+), a high-ionic-strength solution, and resuspended at a concentration of 1 million cells per mL in CAS+. 10% of EQ Four Element Calibration Beads were added to the cells immediately before acquisition. Sample acquisition and data normalization were made on the CyTOF XT mass cytometer and CyTOF software version 8.0.14050 (Fluidigm, Inc Canada) at the “Plateforme de Cytométrie de la Pitié-Salpetriere (CyPS).” An average of 500,000 events were acquired per sample. Dual count calibration, noise reduction, cell length threshold between 10 and 150 pushes, and a lower convolution threshold equal to 12 were applied during acquisition. Data cleaning was performed using Maxpar Pathsetter software v2.0.45. 4 parameters (center, offset, residual and width) were used to resolve ion fusion events (doublets) from single events using the Gaussian distribution generated by each event.2 After data cleaning, the program produces new FCS files consisting of only intact live single cells.

Cleaned FCS files were loaded in R using the flowCore package. FCS files from all samples were concatenated into a SingleCellExperiment object. Clustering was performed using the cluster function from the CATALYST package to identify 60 initial clusters. Clusters were manually identified based on marker expression following Fluidigm Maxpar Immune Profiling manufacturer instruction. Clusters were merged into cohesive cell populations, resulting in 26 final cell types (including 3 groups of contaminants that were removed). Kinetics of cell type proportions over time was plotted using the ggplot2 package.

scRNA-Seq Libraries and Sequencing

ScRNA-seq experiments were performed at the “Labtech SingleCell@Imagine”, in 3 batches for the SAVI dataset, and 3 batches for the IFN dataset. 8 libraries of the STING1 batch were generated using Chromium Single Cell 3′ Library & Gel Bead Kit v.2 (10× Genomics) according to the manufacturer's protocol, while the remaining 9 libraries (STING2 and STING3) were generated using Chromium Single Cell 3′ Library & Gel Bead Kit v.3 (10× Genomics). Briefly, cells were counted, diluted at 1,000 cells/μL in PBS+0.04% and 20,000 cells were loaded in the 10× Chromium Controller to generate single-cell gel-beads in emulsion. After reverse transcription, gel-beads in emulsion were disrupted. Barcoded complementary DNA was isolated and amplified by PCR. Following fragmentation, end repair and A-tailing, sample indexes were added during index PCR. All libraries were sequenced on a Novaseq (Illumina). The 8 purified libraries generated from the v2 kit were sequenced with 26 cycles of read 1, 8 cycles of i7 index and 98 cycles of read 2, while the 9 libraries generated with v3 were sequenced with 28 cycles of read 1, 8 cycles of i7 index and 91 cycles of read 2.

The 24 scRNA-seq libraries of the IFN dataset were generated using Chromium Single Cell Next GEM 3′ Library & Gel Bead Kit v.3.1 (10× Genomics) according to the manufacturer's protocol, following the same steps as in the SAVI dataset. The purified libraries were sequenced on a Novaseq (Illumina) with 28 cycles of read 1, 8 cycles of i7 index and 91 cycles of read 2.

Bioinformatics Analysis of scRNAseq Data

Sequencing reads were demultiplexed and aligned to the human reference genome (hg38), and counted using the CellRanger Pipeline v6.0. Unfiltered RNA UMI counts were loaded into Seurat v4 for quality control, data integration and downstream analyses. Apoptotic cells and empty sequencing capsules were excluded by filtering out cells with low number of features (Filters were defined based on the batch: STING1: nFeature <300; STING2 and STING3<500; IFN-stim <750) or a mitochondrial content higher than 20%. Data from each sample were normalized using sctransform, before batch correction using Seurat's FindIntegratedAnchors, on the 3,000 most variable features. For the IFN-β dataset, for computational efficiency, integration anchors were determined with a canonical correlation analysis, using all unstimulated samples as reference. For the SAVI dataset, to avoid over-aligning samples, a reciprocal principal component analysis (PCA) was used to find integration anchors. On the resulting integrated datasets, we computed the PCA on the 3000 most variable genes, before computing a UMAP (on 20 PCs for SAVI and 30 PCs for IFN-stimulation). Community detection was performed using the graph-based modularity-optimization Louvain algorithm from Seurat's FindClusters function with a 1.2 resolution. Cell types labels were assigned to resulting clusters based on a manually curated list of marker genes as well as previously defined signatures of the PBMC subtypes.⁵⁸ Despite filtering for high quality cells, 11/34 clusters in the SAVI dataset and 7/36 in the IFN-β dataset stood out as poor quality clusters and were removed from further analysis. They represented low UMI cells, dying cells, cycling cells, doublets, or contaminants (progenitor cells, basophils, platelets or megakaryocytes). A total of 112,060 and 115,503 cells were kept in the SAVI and IFN-β datasets respectively. Transcriptomic signatures scores were calculated using Seurat's AddModuleScore function. Differential expression testing was conducted using the FindMarkers function in Seurat, with default Wilcoxon testing. P-values were controlled using Bonferroni correction. Genes with an absolute log (fold-change) ≥0.25 and an adjusted p-value≤0.05 were selected as differentially expressed. Pathway analysis was performed using both the Ingenuity pathway analysis v57662101 software (IPA (QIAGEN Inc.) and EnrichR^{59,60}. Heatmaps were extracted from the comparison module in IPA. Pathways with an absolute z-score lower than 2 or a Bonferroni-Hochberg corrected p-values higher than 0.05 were filtered out. The TRRUST transcription factors 201921 used for the transcription factors enrichment analysis was performed using Enrich R.

The cell cycle phase was defined using the CellCycleScoring function of Seurat V4, with the default gene lists and adjusting the parameters to better reflect expected proportions of CTRL cells in each phase of the cell cycle.

Cell-to-cell interactions were predicted using the ICELLNET framework²⁷. We filtered out filtered the gene expression matrix to only keep the genes that were expressed in at least 5% of the cells in at least one of the clusters. We combined together cluster 5 and cluster 17 into a single classical monocyte group and considered this group as the “central cells”. Each cluster of CD4⁺ or CD8⁺ T cell was considered as the “partner cells”. In the SAVI dataset, the predictions were performed separately for the CTRL, the SAVI, and the SAVI_treated groups. In the IFN-β dataset, the predictions were performed separately for each timepoint.

### Cytokine Measurements

The plasma samples and conditioned media from in vitro stimulated PBMC were analyzed by proximity extension assays (PEA), a targeted, antibody-based, proteomics method. We used the PEA Olink Target96 Inflammation panel which targets 92 biomarkers. Briefly, 2 DNA-labeled antibodies bind to a targeted protein, followed by hybridization of the oligonucleotides when the antibodies come in close proximity to each other. Adding DNA polymerase to the sample generates a unique PCR target sequence, which is then detected and quantified using a quantitative PCR (qPCR) readout (BiomarkHD™, Data collection and Real Time PCR analysis software 4.7.1, Fluidigm).

The Olink PEA Quality control process consists of technical controls to monitor the performance of all 3 steps of the assays (immunoreaction, extension, and amplification/detection) as well as the individual samples. Internal controls are spiked into each sample to provide a non-human assay control, a positive extension control consisting of an antibody coupled to a unique DNA-pair and, finally, a positive detection control based on a double stranded DNA amplicon. In addition, each experimental run includes a control strip with control samples used to estimate the accuracy of protein detection and quantification (intra- and inter-coefficient of variation). A negative control (buffer) run in triplicate is used to set background levels and calculate limit of detection (LOD), a plate control (plasma pool) is run in triplicate for plate normalization, and a sample control (reference plasma) is included in duplicate to estimate CV between runs.

Data are presented as NPX (Normalized Protein expression) values. NPX is Olink's relative protein quantification unit on log 2 scale. Data generation of NPX (obtained using Olink NPX Manager 3.3.2.434) consists of normalization to the extension control (known standard), log 2-transformation, and level adjustment using the plate control (plasma sample).

Serial dilutions of samples are performed to determine a potential Hook effect (observed when there is an antigen excess relative to the reagent antibodies, resulting in falsely lower values). Conditioned media are tested at 1:1, 1:4 and 1:10 dilutions for Target96 Inflammation. Plasma samples are not diluted.

For plasma cytokine measurement, differential protein expression was performed using Multiple comparisons parametric test (TIBCO® Spotfire® v.10.3.2.7) with Benjamini-Hochberg (BH) False Discovery Rate adjustment. For the IFN-β dataset, pairwise analysis by donor was performed by timepoint. For patient samples, SAVI samples were compared to healthy donors. An adjusted p-value<0.05 was considered statistically significant.

### Exome Analysis

The exome sequencing used in this study has been described by Jeremiah et al¹. Exome sequencing was performed by the Centre National de Génotypage, Institut de Génomique, CEA. After quality control by the DNA Bank Laboratory, genomic DNA (3 μg) was captured using in-solution enrichment methodology (Human All Exon v5-50 Mb, Agilent Technologies, CA, USA). Library preparation and exome enrichment protocol (~20,000 targeted genes) was performed on an automated platform, using NGSx (Perkin Elmer Inc, MA, USA) and Bravo (Agilent Technologies, CA, USA) robots respectively, according to manufacturer's instructions (SureSelect, Agilent Technologies CA, USA). After normalization and quality control, exome enriched libraries were sequenced on a HiSEQ 2000 (Illumina Inc., CA, USA) as paired-end 100 base reads. Sequencing was performed in order to provide a mean cover of at least 60 to 70× for each sample. Image analysis and base calling was performed using Illumina Real Time Analysis (RTA) Pipeline. Sequence quality parameters were assessed daily throughout the 12 days sequencing run. Sequences were aligned to the reference human genome hg19 using the Burrows-Wheeler Aligner. Downstream processing was carried out with the Genome Analysis Toolkit (GATK), SAMtools, and Picard, following documented best practices (http://www.broadinstitute.org/gatk/guide/topic?name=best-practices)

We searched for SNPs in the 85 genes of the UPR signature, to extract the SNPs found in less than 5% of the general population. No other filter was used.

We generate a HOPE report (https://www3.cmbi.umcn.nl/hope/)³⁴ for the p.P649T mutation in PERK (encoded by EIF2AK3), by using PERK uniprot identifier Q9NZJ5.

### Generation of the ADUS100 Dataset

Human PBMC samples were obtained from Promocell (C-12907 promocell, lot 434Z036). Briefly, after thawing and a quality control on cell viability (>90%), a minimal of of 2*10⁶ cells cells were incubated either with or without ADUS100 (3 mM final concentration), a STING agonist, during 4 hours in RPMI1640 medium complemented with 10% heat inactivated fetal bovine serum. In some conditions, cells were pre-incubated 15 min with a JAK1-2 inhibitor (baricitinib, 10 mM final concentration) prior and during ADUS100 exposure. After 4 hours, cells were washed with PBS without Ca/Mg containing 0.04% BSA and a minimum of 6,105 harvested for single cell analysis (10× Genomics). Libraries were generated using Chromium Single Cell 3′ Library & Gel Bead Kit v.2 (10× Genomics) according to the manufacturer's protocol. Libraries were sequenced on a Novaseq (Illumina), with 26 cycles of read 1, 8 cycles of i7 index and 98 cycles of read 2.

### Quantification and Statistical Analysis

Statistical tests for cellular composition analysis in both the CyTOF and scRNA-seq datasets were performed in R v3.6.1. Kruskal-Wallis test followed by post-hoc multiple comparison Dunn's test was applied to assess differences in cell population proportions (*: p≤0.05; **: p≤0.01; ***: p≤0.001).

### Results:

Circulating Effector Lymphocytes are Decreased in SAVI, Including MAIT, γδT Cells, NK Cells, Effector CD4⁺ and CD8⁺ T Cells

To better understand the molecular physiopathology of SAVI patients, we profiled at the single-cell transcriptomic level a dataset composed of PBMCs from five SAVI patients carrying different gain-of-function mutations of STING, as well as seven healthy donors (CTRLs) (data not shown). All patients were previously described^{1,3,15,18,21}. We collected blood samples before JAK-inhibitor treatment for all patients (SAVI group), and samples post-JAK-inhibition for four of them (SAVI_treated group). These four patients were treated with the JAK-1/2 inhibitor Ruxolitinib for a duration of 1 to 52 months (4 years and 4 months), and one was later switched to the JAK-1/3 inhibitor, Tofacitinib, for 2 months. For this patient, a blood sample was collected after each treatment.

In parallel, we aimed to better determine the contribution of type I IFN secretion to the transcriptomic profile of SAVI patients. To do so, we analyzed PBMCs from three healthy donors (donors D34, D38 and D39), following in vitro stimulation with human recombinant IFN-β at different timepoints, using the same single-cell approach (data not shown). Response to IFN-β was controlled by following the expression of 6 ISGs commonly used to evaluate type I IFN response in patients²² (data not shown).

After quality control, integration, and unsupervised clustering of each dataset, we obtained 23 cell populations in the SAVI dataset and 29 in the IFN-β dataset (data not shown). We compared cell population proportions in SAVI patients to CTRLs (data not shown), and confirmed the previously described loss of effector CD4⁺ and CD8⁺ T cells¹⁴ as well as NK cells²³ (particularly in NK CD56^{bright} cells). In addition, we observed a drastic reduction in the proportion of MAIT and γδ T cells in SAVI patients (data not shown). We did not observe significant differences in cell type proportions before and after JAK-inhibitor treatment. These observations were subsequently validated by flow cytometry for the SAVI dataset and by Cytometry by time of flight (CyTOF) for the IFN-β dataset (data not shown). Of note, no decrease of effector T cells was observed in healthy PBMCs challenged in vitro with type I IFN, arguing that type I IFN signaling does not impact cell type proportions within 36 hours of stimulation (data not shown).

SAVI Patients Display Strong Type I IFN Response and NF-κB Activation, with Cell Stress, Death, and Modulation of Actin and Integrin-Related Pathways

STING activation triggers NF-κB signaling and type I IFN production²⁴. Therefore, we sought to evaluate these pathways in the immune cells of SAVI patients at the single-cell level. Response to type I IFNs was increased in all PBMC cell types in SAVI patients, with the strongest induction observed in monocytes and DCs (data not shown). This type I IFN response is partially corrected following JAK-inhibitor treatment, with the mean signature score being lower in treated patient compared to untreated, but remains higher than in CTRLs (data not shown). The type I IFN response is also observed after in vitro stimulation with human recombinant IFN-β, with a robust induction as soon as one hour of stimulation, peaking after 2 h (data not shown). Type I IFN response is also the strongest in monocytes and DCs of the IFN-β dataset, specially at later time points. The transcriptomic signature of NF-κB activation follows the pattern of type I IFN response (data not shown), with the strongest induction in monocytes and DCs of SAVI patients. After JAK-inhibitor treatment, NF-κB activation is back to CTRL levels. In the IFN-β-stimulated PBMC dataset, NF-κB activation is also induced by IFN-β as soon as one-hour of stimulation but continues to increase until 36 h particularly in monocytes and DCs (data not shown).

To explore the dysregulated pathways in immune cells of SAVI patients, we used an approach based on differentially expressed genes (DEGs) between untreated SAVI and CTRLs in each major cell population, including the CD4⁺ T cells, CD8⁺ T cells, γδ T cells, MAITs, NKs, B cells and monocytes and DCs. We observed in most cell types an enrichment in pathways involved in response to IFNs or linked to inflammation (data not shown). In the SAVI dataset, the pathways related to IFN and inflammation were downregulated by JAK-inhibitor treatment but remained higher than in CTRLs (data not shown). IFN-related and inflammation-related pathways were also found to be activated in PBMCs treated with IFN-β for 36 h (data not shown). Cell death pathways were enriched in both SAVI patients compared to CTRLs, and in PBMCs stimulated with IFN-β for 36 h compared to unstimulated cells. JAK-inhibitor treatment in patients also seemed to partially correct the induction of these cell death-related pathways (data not shown). In addition, we observed in SAVI patients an enrichment in pathways related to leukocyte trafficking and actin signaling. Although actin/integrin-related pathways are predicted to be inhibited in all cell types in SAVI patients, in healthy PBMCs 36 h post-IFN-β stimulation, these pathways are predicted to be activated in T cells and B cells, while inhibited in monocytes and NK cells (data not shown). Additionally, when looking both in SAVI patients and in vitro stimulated PBMCs, we observe a robust modulation of pathways that could be linked to the integrated stress response (ISR), with downregulation of eIF2 signaling and oxidative phosphorylation and an upregulation of the unfolded protein response (UPR). In the IFN-β dataset, this response was inhibited in a time-dependent manner, with the EIF2 signaling pathway being inhibited as soon as 2 h, with the lowest prediction score at 36 h. Meanwhile, the UPR was induced as soon as 4 h with increasing levels of induction throughout the time course (data not shown). Analyses of transcription factors highlighted a majority of common upstream regulators in both datasets, with some members of the Signal Transducer and Activator of Transcription (STAT), interferon regulating factor (IRF), and NFKB families being significantly predicted as regulating the changes observed in both datasets. (data not shown).

T Lymphocytes of SAVI Patients Present an Hyperactivated, Senescent and Apoptotic Phenotype, Associated with IFN-β Signaling

Imbalance between naïve and effector T cells, along with proliferation defects, have previously been described in SAVI patients¹⁴. To improve our knowledge of transcriptomic dysregulations in T cells from SAVI patients, we performed differential analysis between untreated SAVI patients and CTRLs in each cluster of the CD4⁺ and CD8⁺ T cell compartments. As expected, QIAGEN Ingenuity Pathway Analysis (IPA) revealed a strong enrichment of inflammatory response genes (data not shown). It also pointed to the enrichment of genes involved in actin and integrin-related pathways, and particularly the leukocyte extravasation signaling pathway (data not shown). Several pathways of this category, including RAC signaling and integrin signaling, were particularly decreased in effector cells. We hypothesized that this may reflect an impaired lymphocyte trafficking machinery, which was supported by the decrease of a lymphocyte trafficking signature in T cells of SAVI patients, and particularly in effector T cells (data not shown). This signature was also decreased in effector T cell clusters after IFN-β challenge of PBMCs from healthy donors (data not shown), suggesting that type I IFNs may cause impaired T cell trafficking in SAVI patients.

As T cells of Sting^{N153S/+} mice were shown to undergo spontaneous activation¹¹, we next examined the expression of the early activation marker CD69. T cells of patients displayed a strong increase in the mRNA level of CD69 (data not shown), mostly observed in the clusters we defined as naïve CD4⁺ and CD8⁺ versus effector T cells (data not shown). CD69 expression was also induced by IFN-β challenge at early time points but returned to basal levels between 4 and 12 h post stimulation (data not shown). While the early activation marker remains highly expressed in SAVI and is rapidly controlled in IFN-β-stimulated cells, a global T cell activation signature is increased in both SAVI patients, and IFN-β-challenged cells (data not shown). This activation signature is markedly increased in the naïve cells of SAVI while the activation level stays similar in effector T cells of SAVI patients and CTRL (data not shown). IFN-β stimulates the activation of all clusters of T cells in the IFN-β-challenged PBMCs (data not shown).

The high activation status of naïve T cells associated with a lower proportion of effector cells in SAVI prompted us to hypothesize that naïve T cells failed to expand and differentiate into effector cell upon activation in SAVI. We evaluated a senescence signature as well as the proportion of cells in the Growth and Mitotic phases (G2/M) of the cell cycle (data not shown). T cells of SAVI patients had an increased senescence signature, which was reduced by JAK-inhibitor treatment in naïve T cells (data not shown). A significantly decreased proportion of naïve T cells from SAVI patients in the G2/M phase, partially rescued by the treatment, was also observed, suggesting reduced proliferation capacity compared to CTRL (data not shown). Similarly, in the IFN-β dataset, IFN-β induced increased senescence and decreased proportion of cells in G2/M phase, even though the proportion of cell in the G2/M phase is back to normal after 36 h (data not shown). After showing increased activation and senescence, we evaluated if the cells were apoptotic, and revealed increased transcriptomic signature of apoptosis, particularly in naïve CD4⁺ and CD8⁺ T cells in SAVI patients, restored by JAK-inhibitor treatment (data not shown). Enrichment of genes involved in necroptosis was also observed in pathway analysis (data not shown). The IFN-β-treated cells showed a sharp increase in apoptosis signature after 2 h of treatment, and the signature is decreased afterwards (data not shown). Other cell death pathways are also induced by IFN-β-challenge, such as necroptosis and death receptor signaling (data not shown).

Taken together, these data suggest that T cells, identified as naïve based on a set of well-known typical markers, are highly activated in SAVI but could have reduced ability to proliferate and differentiate into effector cells, ultimately leading to cell death.

### Identification of Disease-Associated Monocytes in SAVI Patients, Presenting Hyperinflammation and Transcriptomic Signature of Integrated Stress Response

As the type I IFN response and the NF-κB signaling signatures were predominantly detected in monocytes and dendritic cells, we next sought to examine these populations in greater details. In addition to the decreased percentages of effector T and NK cells (data not shown), we observed a shift in the distribution of classical monocytes, without changes of global monocytes proportions (data not shown). One cluster, cluster 17, that we named disease-associated cluster, is composed at 85% of cells from patients, with 66.7% of cells from patients before JAK-inhibitor treatment, and 18.3% of cells belonging to samples obtained following treatment (data not shown). In this disease-associated monocytic cluster, in addition to markers of classical monocytes (data not shown), we observed that both the type I IFN response score and the NF-κB activation score were elevated compared to the classical monocytes (Cluster 5), and that non-classical monocytes from SAVI patients were also high for both signatures (data not shown). We evaluated the expression of type I and type III IFNs, which were all increased in SAVI patients compared to CTRL and partially decreased after JAK-inhibitor treatment (data not shown). In SAVI patients, we observed that disease-associated monocytes were the main source of IFN-β, which was also expressed to a lesser extent by classical and non-classical monocytes (data not shown). To better understand the molecular mechanisms underlying the disease-associated cluster, we performed differential expression analysis between the disease-associated monocytes of SAVI patients (cluster 17) and their classical monocytes counterparts (cluster 5). We observed 984 DEGs (data not shown), with the upregulation of key players involved in the integrated stress response (ISR), cell activation and transcripts of IFN and proinflammatory cytokines, along with downregulation of ribosomal genes. Pathway analysis revealed a strong enrichment of genes negatively regulating the EIF2 signaling pathway, which can be associated to an increase of the ISR²⁵ (data not shown). To validate the upregulation of the ISR, we looked at the mRNA level of PPP1R15A, which codes for the ISR marker GADD34. PPP1R15A expression reached the highest level in the disease-associated monocytes in SAVI (data not shown). An unfolded protein response (UPR) signature, known to contribute to the ISR, obtained from Raich et al²⁶, followed a similar pattern with a high score in patients' disease-associated monocytes. Of note, non-classical monocytes expressed some level of PPP1R15A or UPR. JAK-inhibitors seem to partially reduce these pathways in both non-classical and disease-associated clusters (data not shown). In the dataset of IFN-β stimulated monocytes both PPP1R15A and the UPR signature were also increased after 36 h of stimulation (data not shown). Among the other pathways upregulated in this disease-associated cluster, we also report pathways linked to response to IFN, production of inflammatory cytokines, and cell death along with an enrichment of genes involved in oxidative phosphorylation (data not shown). Overall, this disease-associated cluster seems to correspond to classical monocytes that display a high inflammation and ISR which could be associated with high STING activation.

Inference of Cell-to-Cell Communication Predicts that Hyperinflammatory Monocytes in SAVI could Drive Hyperactivation and Death of Effector T Cells

To gain a better understanding of the interactions between disease-associated classical monocytes and T cells of patients, in which we identified major dysregulations at the transcriptomic level (data not shown), we sought to infer monocytes-to-T cell communications. To this end, we used the ICELLNET framework²⁷, which infers a score of cell-to-cell communication based on the ligands expressed by a sender cell type (here, both clusters of classical monocytes; cluster 5. Classical monocytes, and cluster 17. Disease-associated monocytes), and the receptors expressed by a receiving cell type (here, T cells; data not shown). We applied this framework to our SAVI dataset to infer the communication between classical monocytes (including the disease-associated cluster) and each CD4⁺ and CD8⁺ T cell cluster in the SAVI group and the CTRL group separately (data not shown). We reproduce a similar analysis on our IFN-β-stimulated PBMC dataset for each time point (data not shown), which allowed us to decipher IFN-dependent and IFN-independent predicted interactions once compared to the SAVI dataset. Hierarchical clustering of the T cells clusters based on their communication score with monocytes showed a clear separation between clusters from untreated SAVI patients and healthy donors (data not shown). Based on our previous observations, where we detected transcripts of IFN genes in monocytes of SAVI patients (data not shown), we were not surprised to have a predicted increase of type I IFN-IFNAR interactions in SAVI patients, although this interaction is not predicted in the IFN-β dataset (data not shown). This observation most probably reflects the drastic expression of IFN transcripts in the monocytes of SAVI patients. Other proinflammatory cytokines including CXCL9, CXCL10, and CXCL11 with their common receptor CXCR3, as well as the pair IL-6/IL-6R, are predicted to have increased interactions in a type I IFN-dependent manner (data not shown). In contrast, the increase of IL-12 and IL-27 interactions with their receptors seems independent of type I IFNs as it is absent from the predictions in the IFN-β dataset.

Strikingly, the strongest predicted interaction in SAVI patients is the pair CCL19/CCR7, which plays an essential role in T cell activation²⁸. This communication is not predicted to occur in healthy PBMCs treated with IFN-β, nor in SAVI patients after JAK-inhibitor treatment (data not shown), arguing for a mechanism independent of type I IFN production. Checkpoint interactions, such as CD80/CD28, Nectin2/CD226, Nectin2/TIGIT, and Nectin2/CD96, involved in T cells stimulations²⁹, are also increased in both SAVI patients and following IFN-β stimulation but should be interpreted with caution as monocytes are not professional antigen presenting cells. Moreover, we are predicting enhanced crosstalk between LAG3 and its ligand LGALS3 and MHC class I molecules in SAVI patients, corresponding to the strongest induction predicted in the IFN-β dataset (data not shown). We also observed in SAVI patients and IFN-β stimulated cells increased communications involved in apoptosis induction with TNFSF10/TNFRSF10B and LGALS1/CD69 (data not shown)^{30,31}. In CD8⁺ T cell interactions with monocytes, the pair MICA/KLRK1 was predicted to be increased in SAVI patients. MICA is a stress-induced ligand belonging to the MHC-I family and known to lead to cell lysis when bound to its receptor KLRK1/NKG2D³² (data not shown). The same interaction is also predicted to be strongly induced by IFN-β-stimulation exclusively in CD8⁺ T lymphocytes. Overall, these data point towards a role of the disease-associated monocytes in favoring T cells chronic activation and death.

### Circulating Cytokines in the Plasma of SAVI Patients as Biomarkers of the Disease

As several cytokines/chemokines were seen upregulated in SAVI patients in transcriptomics data, we evaluated the level of 96 inflammation-related proteins in the plasma of SAVI patients and associated CTRLs. We confirmed the previously known increase of IL-6¹⁶, and we observed increased plasmatic levels of CXCL10 and CCL19 (data not shown), which were predicted to play a role in monocytes-to-T cell communications (data not shown). Other proinflammatory cytokines, such as CCL3, CCL4, CCL7, and CCL8, were also increased in the plasma of SAVI patients compared to CTRLs (data not shown). The secretory profile of PBMCs stimulated with IFN-β for 36 h was characterized by induction of CXCL10, VEGFA and CCL19 (data not shown). Although IFN-β-stimulation increased secretion of CXCL8, we observed its decrease in SAVI patients, further highlighting IFN-independent mechanisms involved in SAVI disease. We tested the expression of the 12 cytokines increased in the plasma of SAVI patients on the scRNA-seq data revealing a strong role of the disease-associated monocytes in the secretion of several of these cytokines. These results further points to a role of disease-associated monocytes in the SAVI disease, even though the transcripts levels of some of these cytokines were close to the limits of detection (data not shown). Of note, CCL3, CCL4 and IL-6 were among the most upregulated genes in the disease-associated monocytes (data not shown). PBMCs stimulated with IFN-β had a more homogeneous expression of inflammatory cytokines across cell types, in particular monocytes, DCs, and NK cells (data not shown). The specific elevation of type I IFN-independent cytokines, in particular CCL3, CCL4 and IL-6, suggests that they may be used as blood biomarkers of SAVI to aid the differential diagnosis with other type I interferonopathies.

### Proposition of a Transcriptomic Signature Specific of STING Activation and Independent of Response to IFN-β

Our next objective was to identify a transcriptomic signature of STING activation, that may be used to segregate SAVI patients from patients with other type I interferonopathies. As we have pinpointed a group of monocytes specific to the disease with elevated inflammation and ISR, we aimed to establish a transcriptomic signature based on the genes expressed by these cells. The first step therefore consisted in the extraction of the genes specific to the disease-associated cluster (Step 1, data not shown). We obtained 95 genes that are highly upregulated in the disease-associated cluster compared to the other cluster of classical monocytes in SAVI patients (Cluster 17 vs Cluster 5, only on SAVI cells, FDR <0.05, log 2FC >1). One gene, G0S2, was filtered out from the list as its expression was not increased in SAVI compared to CTRLs (Step 2, data not shown). As we aimed to establish a signature that would allow to segregate SAVI patients from patients with other type I interferonopathies, an important step of the gene selection was to remove all genes related to type I IFN. We started by removing all type I IFN transcripts that were found in the list (namely IFNA1, IFNA8, IFNA14, IFNB1, and IFNE) (Step 3, data not shown) and then aimed to remove ISGs. To this end, we explored the genes that were upregulated by IFN-β in each timepoint of the IFN-dataset. We removed 68 genes that were increased by type I IFN in monocytes and DCs in at least one of the timepoints of our dataset (Step 4, data not shown). Consequently, 21 genes remained in the STING activation signature: AC253572.2, ADRB2, ANKRD28, ANXA1, AQP9, AZIN1-AS1, IL1B, JARID2, KLF4, MIR4435-2HG, OTUD1, PLAUR, PTGS2, PTX3, SELENOK, TGIF1, TREM1, TSC22D2, ZBTB43, ZFP36L2 and ZNF503. These genes were mostly associated with TNF-α signaling via NF-κB (data not shown). We confirmed that this signature was specific to the SAVI patients in our patients' dataset, and observed that it was partially reduced in JAK-inhibitor-treated patients (data not shown). We also confirm in our IFN data set that the resulting signature is not increased by type I IFN at any timepoint (data not shown).

To confirm the relevance of this signature in the context of STING activation, and to evaluate whether the signature is indeed type I IFN-independent, we built an in vitro model of STING activation, in which we used PBMCs from a healthy donor, either unstimulated, stimulated with ADUS100 (a STING agonist), or with a combination of ADUS100 and JAK-inhibitor (inhibitor of type I IFN signaling). After performing scRNA-seq, we evaluated STING activation efficiency by measuring the score of type I IFN response and NF-κB activation, which were increased by ADUS100 and controlled by the addition of a JAK-inhibitor (data not shown). We then plotted our STING-associated signature in the monocytes and DCs and observed a strong increase of the signature by ADUS100 stimulation, which remained high when blocking the response to type I IFN (ADUS100+Jak-inhibitor) (data not shown). The elevation of this signature in response to STING activation is restricted to monocyte and DC populations in both the SAVI patients (data not shown) and the ADUS100 stimulated PBMCs (data not shown).

Overall, we propose that this list of 21 genes may be used to direct patients with undetermined type I interferonopathy towards a SAVI diagnosis.

Identification of a Second Mutation in PERK in a SAVI Patient with Low UPR

While analyzing the transcriptomic profiles of the SAVI patients, we noticed several pathways that were differently modulated in one of the patients, P1. We noted in this patient a marked reduction of the UPR, which was largely increased in other patients (data not shown) and is linked to the ISR through their common mediator PERK. Of note, IFN-β stimulation induced a slight increase of the UPR (data not shown). This observation is accompanied by a very low expression of the ISR marker PPP1R15A, which codes for GADD34, in monocytes and DCs (data not shown). Further exploration showed differences in induction of the apoptosis signature and NF-κB signaling, though less drastic than the UPR (data not shown). These differences are even observable when compared to P2, P1's father's homozygotic twin (data not shown). Other pathways however, such as the type I IFN response, are increased in this patient similarly than in other SAVI patients (data not shown). At the clinical level, P1 is the only patient to present with a systemic lupus erythematosus (SLE)-like phenotype1.

The drastic default of UPR induction in P1 compared to the other patients, even to her uncle P2, prompted us to reanalyze the exome sequencing previously performed for this patient¹, focusing specifically on the 85 genes of the UPR signature (data not shown). Exome analysis revealed single-nucleotide polymorphisms (SNPs) in four genes: CTH, VEGFA, ZCCHC8 and EIF2AK3 (data not shown). Mutations in CTH and VEGFA are intronic or in untranslated regions and do not induce STOP codon, and therefore, may not induce a change in protein function. However, mutations in ZCCHC8 and EIF2AK3 induces core changes in amino acid chain and may be of importance. While the STING p. V155M mutation was inherited from the father, both mutations are inherited from the healthy mother suggesting the mutations may play a role in the transcriptomic differences observed between this patient and her uncle P2 (data not shown). The SNP in ZCCHC8 induces a p.G551D. Interestingly, the protein encoded by this gene was previously reported to interact with STING in mouse bone marrow-derived DCs 33. However, even though this mutation induces a change in charge, it is fairly common in the population, with a prevalence of 0.00405. This may suggest limited pathogenicity by itself but may be deleterious when combined with a STING mutation as in P1. The last SNP induces a p.P649T in EIF2AK3, which codes for PERK. This protein was recently shown to be activated upon STING activation through direct interaction between the C-terminal tail of STING and the kinase domain of PERK 20. Once activated, PERK induces the phosphorylation of key components of the ISR. The p.P649T mutation carried by P1 is in the kinase domain of PERK, which carries the ISR-inducing function. This mutation induces a change in residue type from a nonpolar amino acid (Proline) to a polar amino acid (Threonine). We tested this mutant against the HOPE database to gain insight into the impact that this mutation may bring³⁴. The report generated mentions that even though the wild-type residue itself is not conserved, the mutant residue is not among the residue types observed at this position in other homologous sequences, which may indicate a possibly damaging effect to the protein. Additionally, this mutation was never described, which may indicate a deleterious effect. Overall, we highlight two important SNPs in ZCCHC8 and EIF2AK3 which may explain the unique profile of the P1 SAVI patient.

In addition, our results show how single-cell transcriptomics can be leveraged to make new hypothesis regarding patients' genetics.

### Discussion

In the present study, we combine single-cell high-throughput analysis of PBMCs from five SAVI patients before and after JAK-inhibitor treatment with the analysis of in vitro IFN-β-stimulated PBMCs from 1 h to 36 h. We highlight the main pathways dysregulated in the immune cells of SAVI patients, including inflammation, an integrated stress response and cell death. We reveal a default of T cell activation, and pinpoint to a population of disease-associated monocytes. By leveraging cell-to-cell communication prediction, we suggest that these disease-associated monocytes may be responsible for the decreased proportion of circulating effector T cells in the blood of SAVI patients. Together, our findings provide a deeper understanding of the SAVI disease, and we provide a transcriptomic signature of STING activation.

T cell lymphopenia is a clinical hallmark of SAVI^{1,14}. However, studies that have sought to investigate the underlying mechanisms have done so by combining different T cell stages together, such as naïve and effector CD4⁺ or CD8⁺ T cells. We have confirmed the drastic reduction of the frequency of effector T cells in our cohort of SAVI patients. By exploring other T cells subsets found in the blood of SAVI patients at the single cell level, we highlight a unique phenotype in clusters that are defined by transcriptomic markers as naïve T cells. We reveal that these T cells designated as naïve seem to be maintained in an early activation state as measured by CD69 expression. This early T cell activation is observed as soon as 1 h after IFN-β stimulation, but strongly downregulated after 4 h. These results are in agreement with previous reporting suggesting that T cells carrying a gain-of-function STING1 mutation may be constitutively activated as a result of homeostatic reactivity to self-antigen^{35,11}. Additionally, we could hypothesize that T cells are activated in an antigen-independent manner by inflammatory cytokines including type I IFN³⁶. While activation should prompt T cell proliferation, we report increased activation of the senescence pathways in naïve T cells of SAVI patients, associated with a decrease in the proportion of cells reaching the G2M phase of the cell cycle. While our IFN-β stimulation dataset shows decreased passage of T cells into G2M phase only transiently, several studies have highlighted the ability of chronic IFN-β to trigger senescence^{37,38}. These results concur with the antiproliferative role of STING¹⁴, as well as its pro-senescence^{10,39} and pro-apoptotic¹² roles. Altogether, our results could suggest that the decrease of effector T cells may result from the altered naïve T cell compartment. We propose that naïve T cells could lose their ability to transition into an effector state due to an hyperactivation state combined with premature senescence and apoptosis.

In our study, we identify a disease-associated cluster composed of 85% of patient cells suggesting that these cells may be disease-associated. We identify this cluster as classical monocytes origin, and due to the increased expression of activation markers, we conclude that this cluster may represent activated inflammatory monocytes, that may represent classical monocytes with a high activation of STING. These disease-associated monocytes have elevated GADD34 gene expression, an increased UPR signature, and decreased ribosomal genes expression, which altogether imply the activation of the ISR. IFN-β overexpression may play a role in the onset of this response, as we observe its increase in PBMCs treated with IFN-β for 36 h. While our results do not allow us to conclude on the exact mechanism driving the increase of ISR, the recent discovery of the direct activation by STING of an ISR mediator, PERK, suggest that it could be mediated by PERK²⁰. PERK is an ER-stress sensor that, like inactivated STING, is found on the ER membrane. Upon unfolded protein sensing, PERK phosphorylates eIF2α, thereby triggering the ISR. Its direct activation by STING, independently of unfolded proteins, has recently been demonstrated in human cell lines and in mice²⁰. These observations suggest that elevated UPR in this cluster reflects a STING-induced activation of PERK. However, as PERK is not the only trigger for UPR, we cannot exclude that other mechanisms are involved in high UPR in these disease-associated monocytes.

Intriguingly, we highlighted the patient P1 that, as opposed to other patients, presented a low UPR signature. This SAVI patient is the only one in our cohort who had lupus-like manifestations. Exome analysis of the 85 genes of the UPR signature revealed a missense p.P649T mutation in PERK in this patient, inherited from her healthy mother. Prolines are important residues for protein backbone⁴⁰, often found in tight turns in protein structures. The P649 residue is found between an α-helix and a β-sheet in the cytoplasmic kinase domain. Substitution with a threonine may change the structure of the protein and affect the kinase activity. This residue lies in the kinase domain of PERK, which is responsible for eIF2α phosphorylation, a key event for the initiation of the ISR. This mutation is predicted to be highly deleterious. In addition, this mutation has never been described, suggesting that this extremely rare variant may be highly detrimental. We found some cells in the P1 patient with high UPR and identified these as plasmablasts. Interestingly, it has been shown that plasmablasts are characterized by high UPR due to their essential secretory role, but in a PERK-independent manner^{41,42}. The fact that plasmablasts of the PERK-mutated patient retain their ability to activate the UPR, further points to a PERK-dependent mechanism for the activation of the UPR in disease-associated monocytes. We concluded that the unique UPR defect in the P1 patient may be associated with the SNP located in the PERK kinase domain. To go further, the kinase domain of PERK is responsible for eIF2α phosphorylation, a key event for the initiation of the ISR which overall strengthen the importance of PERK mediated ISR in disease-associated monocytes of SAVI patients.

In many autoinflammatory diseases, the cellular source of IFN is still debated. While it could be assumed that pDCs, which are professional IFN-producers, are the main source of type I IFN in inflammatory diseases, they have also been suggested to gradually lose their ability to produce IFN-α in lupus-prone mice^{43,44}. Here, we report that several cell populations express transcripts of type I IFNs in SAVI patients: classical monocytes, non-classical monocytes, and disease-associated monocytes. These results partially corroborate the previous finding that pDCs and monocytes expressed IFN-α proteins in SAVI patients⁴⁵. Our data extend this knowledge by indicating that disease-associated monocytes show the highest production of IFN-β.

By leveraging an expert-curated database of intercellular communications to infer monocytes-to-T cells interactions²⁷, we provided clues that in human hyperinflammatory monocytes could promote activation of T cells, as well as cell death. This method revealed the establishment of T cell-activating signaling in SAVI patients. Indeed, we note the increase of CCL19/CCR7 and LGALS1/CD69, which play a role in T cell activation^{28,30}. Several of the communications inferred involved checkpoint interactions. While monocytes are not antigen-presenting cells (APCs) themselves, recent work has suggested the ability of activated monocytes to activate CD8⁺ T cells⁴⁶. Additionally, they are the precursor of monocyte-derived macrophages and dendritic cells, which are APCs⁴⁷. The increase of transcripts of the presenting machinery in blood monocytes may reflect the enhanced capacity of these monocytes-derived presenting cells to effectively activate T cells. Additionally, we note several communications that trigger T cell death, such as TRAIL(TNFSF10)/TNFRSF10B⁴⁸ or MIKA/KLRK1³². These data point towards a potent role of the monocytes to prime T cells towards activation and cell death. Although these interactions were predicted at the transcriptomic level, several of the cytokines involved in the predicted monocytes-to-T cells communications were also elevated at the protein level in the plasma of SAVI patients, confirming that the predicted interactions are possible. We have also shown that most of the cytokines that we have found increased were likely secreted by the disease-associated monocytes.

Further studies are necessary to investigate the contribution of other cell types that were not included in this work, such as neutrophils or epithelial cells, which may also play a role in the secretory profiles of the blood of SAVI patients. Nonetheless, our results converge towards a central role of these disease-associated monocytes in SAVI, particularly in the priming of T cells towards hyperactivation and apoptosis.

To further evaluate the relevance of these cells in the pathogenesis of SAVI, we suggest that their presence should be investigated in the lungs of SAVI patients. Indeed, monocytic infiltrate has been observed, and it is conceivable that it is composed of the disease-associated monocytes identified in the blood. The role of monocytes and monocyte-derived macrophages is increasingly recognized in pulmonary fibrosis⁴⁹⁻⁵². Additionally, the integrated stress response, a strongly induced pathway in these monocytes, has also been proposed as an important driver of pulmonary fibrosis⁵³, which further supports the hypothesis that the disease-associated monocytes may drive the pulmonary damage in SAVI patients. If these disease-associated monocytes were indeed retrieved in the lungs of SAVI patients, we propose that they may constitute an interesting indication for treatment monitoring. Other potential targets of interest in SAVI are PERK and the ISR, which are the targets of compounds under development for other diseases⁵⁴.

Four patients were treated with JAK-inhibitors (mainly Ruxolitinib, a JAK-1/2 inhibitor). While JAK-inhibitors are beneficial to SAVI patients, they do not allow for complete remission, and patients with advanced lung disease prior to treatment show little to no amelioration³. Here, we have assessed the impact of the treatment on each pathway evaluated, and our data support an incomplete remission of the patients. In T cells, we indicate that while JAK-inhibitor treatments seem to correct the senescence and apoptosis observed in naïve T cells, they do not allow to restore the proportion of T cells to normal levels. Notably, Tofacitinib was recently suggested to inhibit T cell proliferation⁵⁵ and further work is needed to evaluate if JAK-1/2 inhibitors also induce the same effect.

The current diagnosis of type I interferonopathies, including SAVI, relies on clinical evaluation followed by measurement of the level of expression of 6 interferon-stimulated genes in PBMCs of suspected patients. This approach is common to all type I interferonopathies, and subsequent exome sequencing (an expensive approach) is required to pinpoint a specific gene¹. There is therefore an unmet need for an easy-to-use, differential diagnosis strategy between the different type I interferonopathies. In this work, we have identified at the transcriptomic level a STING-associated signature driven by STING activation, independently of type I IFN. Furthermore, the STING-associated signature may be relevant in the stratification of SLE patients. Indeed, SLE has been shown the represent not one disease, but up to seven group of “sub-diseases”⁵⁶, and STING may be activated in some patients^{33,57}. Thus, we think that our signature may bring new insights into SLE patient stratification. In addition, we noticed the elevation at the transcriptomic and protein level of CCL3, CCL4 and IL-6, and we propose that they be added to the panel 6 ISGs routinely measured. An elevated cytokine expression of these cytokines associated with high ISG score could lead to a suspicion of STING mutation.

In summary, our study represents the first single-cell RNA sequencing analysis performed on SAVI patients. We propose that the loss of effector T cells in SAVI patients could be consequence of an altered ability of naïve cells to differentiate and highlight a pathogenic group of monocytic cells characterized by the elevated integrated stress response. Moreover, we propose a STING-associated signature that would be suitable to better distinguish SAVI from other type I interferonopathy. Additionally, we have leveraged scRNA-seq data to better understand immune cell profile in a unique patient, a step further toward personalized medicine.

### REFERENCES

Throughout this application, various references describe the state of the art to which this invention pertains. The disclosures of these references are hereby incorporated by reference into the present disclosure.
1. Jeremiah, N. et al. Inherited STING-activating mutation underlies a familial inflammatory syndrome with lupus-like manifestations. J Clin Invest 124, 5516-5520 (2014).
2. Liu, Y. et al. Activated STING in a Vascular and Pulmonary Syndrome. New England Journal of Medicine 371, 507-518 (2014).
3. Frémond, M.-L. et al. Overview of STING-Associated Vasculopathy with Onset in Infancy (SAVI) Among 21 Patients. The Journal of Allergy and Clinical Immunology: In Practice 9, 803-818.e11 (2021).
4. David, C. & Frémond, M.-L. Lung Inflammation in STING-Associated Vasculopathy with Onset in Infancy (SAVI). Cells 11, 318 (2022).
5. Dobbs, N. et al. STING Activation by Translocation from the ER Is Associated with Infection and Autoinflammatory Disease. Cell Host & Microbe 18, 157-168 (2015).
6. Ishikawa, H., Ma, Z. & Barber, G. N. STING regulates intracellular DNA-mediated, type I interferon-dependent innate immunity. Nature 461, 788-792 (2009).
7. Abe, T. & Barber, G. N. Cytosolic-DNA-mediated, STING-dependent proinflammatory gene induction necessitates canonical NF-κB activation through TBK1. J Virol 88, 5328-5341 (2014).
8. Yum, S., Li, M., Fang, Y. & Chen, Z. J. TBK1 recruitment to STING activates both IRF3 and NF-κB that mediate immune defense against tumors and viral infections. Proc Natl Acad Sci USA 118, e2100225118 (2021).
9. Tanaka, Y. & Chen, Z. J. STING Specifies IRF3 phosphorylation by TBK1 in the Cytosolic DNA Signaling Pathway. Sci Signal 5, ra20 (2012).
10. Dou, Z. et al. Cytoplasmic chromatin triggers inflammation in senescence and cancer. Nature 550, 402-406 (2017).
11. Wu, J. et al. STING-mediated disruption of calcium homeostasis chronically activates ER stress and primes T cell death. J Exp Med 216, 867-883 (2019).
12. Gulen, M. F. et al. Signalling strength determines proapoptotic functions of STING. Nat

### Commun 8, 427 (2017).

13. Staels, F. et al. Adult-Onset ANCA-Associated Vasculitis in SAVI: Extension of the Phenotypic Spectrum, Case Report and Review of the Literature. Front Immunol 11, 575219 (2020).
14. Cerboni, S. et al. Intrinsic antiproliferative activity of the innate sensor STING in T lymphocytes. Journal of Experimental Medicine 214, 1769-1785 (2017).
15. Picard, C. et al. Severe Pulmonary Fibrosis as the First Manifestation of Interferonopathy (TMEM173 Mutation). Chest 150, e65-e71 (2016).
16. Tang, X. et al. STING-Associated Vasculopathy with Onset in Infancy in Three Children with New Clinical Aspect and Unsatisfactory Therapeutic Responses to Tofacitinib. J Clin Immunol 40, 114-122 (2020).
17. Lin, B. et al. A novel STING1 variant causes a recessive form of STING-associated vasculopathy with onset in infancy (SAVI). Journal of Allergy and Clinical Immunology 146, 1204-1208.e6 (2020).
18. Frémond, M.-L. et al. Efficacy of the Janus kinase 1/2 inhibitor ruxolitinib in the treatment of vasculopathy associated with TMEM173-activating mutations in 3 children. Journal of Allergy and Clinical Immunology 138, 1752-1755 (2016).
19. Gao, K. M., Motwani, M., Tedder, T., Marshak-Rothstein, A. & Fitzgerald, K. A. Radioresistant cells initiate lymphocyte-dependent lung inflammation and IFNγ-dependent mortality in STING gain-of-function mice. Proc Natl Acad Sci USA 119, e2202327119 (2022).
20. Zhang, D. et al. A non-canonical cGAS-STING-PERK pathway facilitates the translational program critical for senescence and organ fibrosis. Nat Cell Biol 24, 766-782 (2022).
21. Munoz, J. et al. Stimulator of Interferon Genes-Associated Vasculopathy With Onset in Infancy: A Mimic of Childhood Granulomatosis With Polyangiitis. JAMA Dermatol 151, 872 (2015).
22. Jeremiah, N. et al. Inherited STING-activating mutation underlies a familial inflammatory syndrome with lupus-like manifestations. J. Clin. Invest. 124, 5516-5520 (2014).
23. Picard, C. et al. Severe Pulmonary Fibrosis as the First Manifestation of Interferonopathy (TMEM173 Mutation). Chest 150, e65-71 (2016).
24. Balka, K. R. et al. TBK1 and IKKε Act Redundantly to Mediate STING-Induced NF-κB Responses in Myeloid Cells. Cell Reports 31, 107492 (2020).
25. Pakos-Zebrucka, K. et al. The integrated stress response. EMBO Rep 17, 1374-1395 (2016).
26. Reich, S. et al. A multi-omics analysis reveals the unfolded protein response regulon and stress-induced resistance to folate-based antimetabolites. Nat Commun 11, 2936 (2020).
27. Noël, F. et al. Dissection of intercellular communication using the transcriptome-based framework ICELLNET. Nat Commun 12, 1089 (2021).
28. Yan, Y. et al. CCL19 and CCR7 Expression, Signaling Pathways, and Adjuvant Functions in Viral Infection and Prevention. Front Cell Dev Biol 7, 212 (2019).
29. Shrager, S. H. & Kiel, C. SnapShot: APC/T Cell Immune Checkpoints. Cell 183, 1142-1142.e1 (2020).
30. Cedeno-Laurent, F. & Dimitroff, C. J. Galectin-1 research in T cell immunity: Past, present and future. Clin Immunol 142, 107-116 (2012).
31. Fulda, S. Tumor-necrosis-factor-related apoptosis-inducing ligand (TRAIL). Adv Exp Med Biol 818, 167-180 (2014).
32. Guerra, N. & Lanier, L. L. Editorial: Emerging Concepts on the NKG2D Receptor-Ligand Axis in Health and Diseases. Frontiers in Immunology 11, (2020).
33. Thim-Uam, A. et al. STING Mediates Lupus via the Activation of Conventional Dendritic Cell Maturation and Plasmacytoid Dendritic Cell Differentiation. iScience 23, 101530 (2020).
34. Venselaar, H., the Beek, T. A., Kuipers, R. K., Hekkelman, M. L. & Vriend, G. Protein structure analysis of mutations causing inheritable diseases. An e-Science approach with life scientist friendly interfaces. BMC Bioinformatics 11, 548 (2010).
35. Kieper, W. C. & Jameson, S. C. Homeostatic expansion and phenotypic conversion of naïve T cells in response to self peptide/MHC ligands. Proceedings of the National Academy of Sciences 96, 13306-13311 (1999).
36. Ramanathan, S. et al. Cytokine synergy in antigen-independent activation and priming of naive CD8+ T lymphocytes. Crit Rev Immunol 29, 219-239 (2009).
37. Frisch, S. M. & MacFawn, I. P. Type I interferons and related pathways in cell senescence. Aging Cell 19, e13234 (2020).
38. Yu, Q. et al. DNA-damage-induced type I interferon promotes senescence and inhibits stem cell function. Cell Rep 11, 785-797 (2015).
39. Yang, H., Wang, H., Ren, J., Chen, Q. & Chen, Z. J. cGAS is essential for cellular senescence. Proceedings of the National Academy of Sciences 114, E4612-E4620 (2017).
40. Betts, M. J. & Russell, R. B. Amino acid properties and consequences of substitutions. in Bioinformatics for Geneticists.
41. Ma, Y., Shimizu, Y., Mann, M. J., Jin, Y. & Hendershot, L. M. Plasma cell differentiation initiates a limited ER stress response by specifically suppressing the PERK-dependent branch of the unfolded protein response. Cell Stress Chaperones 15, 281-293 (2010).
42. Zhu, H. et al. Ufbp1 promotes plasma cell development and ER expansion by modulating distinct branches of UPR. Nat Commun 10, 1084 (2019).
43. Zhou, Z. et al. Phenotypic and functional alterations of pDCs in lupus-prone mice. Sci Rep 6, 20373 (2016).
44. Liao, X. et al. Cutting Edge: Plasmacytoid Dendritic Cells in Late-Stage Lupus Mice Defective in Producing IFN-α. J Immunol 195, 4578-4582 (2015).
45. Rodero, M. P. et al. Detection of interferon alpha protein reveals differential levels and cellular sources in disease. J Exp Med 214, 1547-1555 (2017).
46. Affandi, A. J. et al. CD169 Defines Activated CD14+ Monocytes With Enhanced CD8+ T Cell Activation Capacity. Frontiers in Immunology 12, (2021).
47. Théry, C. & Amigorena, S. The cell biology of antigen presentation in dendritic cells. Curr Opin Immunol 13, 45-51 (2001).
48. Wang, S. & El-Deiry, W. S. TRAIL and apoptosis induction by TNF-family death receptors. Oncogene 22, 8628-8633 (2003).
49. Misharin, A. V. et al. Monocyte-derived alveolar macrophages drive lung fibrosis and persist in the lung over the life span. J Exp Med 214, 2387-2404 (2017).
50. McCubbrey, A. L. et al. Deletion of c-FLIP from CD11bhi Macrophages Prevents Development of Bleomycin-induced Lung Fibrosis. Am J Respir Cell Mol Biol 58, 66-78 (2018).
51. Fraser, E. et al. Multi-Modal Characterization of Monocytes in Idiopathic Pulmonary Fibrosis Reveals a Primed Type I Interferon Immune Phenotype. Frontiers in Immunology 12, (2021).
52. Kessler, N. Monocyte-derived macrophages aggravate pulmonary vasculitis via cGAS/STING/IFN-mediated nucleic acid sensing. 51 (2022).
53. Barabutis, N. Unfolded Protein Response in Lung Health and Disease. Front Med (Lausanne) 7, 344 (2020).
54. Derisbourg, M. J., Hartman, M. D. & Denzel, M. S. Modulating the integrated stress response to slow aging and ameliorate age-related pathology. Nat Aging 1, 760-768 (2021).
55. Kim, D. et al. Targeted therapy guided by single-cell transcriptomic analysis in drug-induced hypersensitivity syndrome: a case report. Nat Med 26, 236-243 (2020).
56. Banchereau, R. et al. Personalized Immunomonitoring Uncovers Molecular Networks that Stratify Lupus Patients. Cell 165, 551-565 (2016).
57. An, J. et al. Expression of Cyclic GMP-AMP Synthase in Patients With Systemic Lupus Erythematosus. Arthritis Rheumatol 69, 800-807 (2017).
58. Monaco, G. et al. RNA-Seq Signatures Normalized by mRNA Abundance Allow Absolute Deconvolution of Human Immune Cell Types. Cell Rep 26, 1627-1640.e7 (2019).
59. Chen, E. Y. et al. Enrichr: interactive and collaborative HTML5 gene list enrichment analysis tool. BMC Bioinformatics 14, 128 (2013).
60. Kuleshov, M. V. et al. Enrichr: a comprehensive gene set enrichment analysis web server 2016 update. Nucleic Acids Res 44, W90-97 (2016).

1. A method for diagnosing a STING-associated vasculopathy with onset in infancy (SAVI) in a subject, comprising:
determining in a sample obtained from the subject an expression level of one or more gene(s) selected from the group consisting of AC253572.2, ADRB2, ANKRD28, ANXA1, AQP9, AZIN1-AS1, IL1B, JARID2, KLF4, MIR4435-2HG, OTUD1, PLAUR, PTGS2, PTX3, SELENOK, TGIF1, TREM1, TSC22D2, ZBTB43, ZFP36L2 and ZNF503;
indicating whether the subject suffers or not from a SAVI based on the expression level of the one or more genes determined in the determining step.

2. The method of claim 1 wherein expression levels higher than a reference value indicate that the subject suffers from a SAVI whereas expression levels below the reference value indicate that the subject do not suffer from a SAVI.

3. The method of claim 1 further comprising discriminating SAVI from another one type I interferonopathy in the subject.

4. The method of claim 3 wherein expression levels higher than a reference value indicate that the subject suffers from a SAVI whereas expression levels lower than the reference value indicate that the subject suffers from another type I interferonopathy.

5. A method of determining whether a subject suffering from a SAVI achieves a response to a therapy comprising:
determining the expression level of one or more gene(s) selected from the group consisting of AC253572.2, ADRB2, ANKRD28, ANXA1, AQP9, AZIN1-AS1, IL1B, JARID2, KLF4, MIR4435-2HG, OTUD1, PLAUR, PTGS2, PTX3, SELENOK, TGIF1, TREM1, TSC22D2, ZBTB43, ZFP36L2 and ZNF503; and
indicating the subject achieves or not a response to the therapy based on the expression level of the one or more genes determined in the determining step.

6. The method of claim 5 wherein levels of expression lower than a reference value indicate that the subject achieves a response to the therapy whereas levels of expression higher than the reference value indicate that the subject does not achieve a response to the therapy.

7. The method of claim 5 wherein the therapy consists of administering to the subject a therapeutically effective amount of a JAK inhibitor.

8. A method of determining whether a subject suffering from a type I interferonopathy is eligible for treatment with a STING antagonist comprising:
determining the expression level of one or more gene(s) selected from the group consisting of AC253572.2, ADRB2, ANKRD28, ANXA1, AQP9, AZIN1-AS1, IL1B, JARID2, KLF4, MIR4435-2HG, OTUD1, PLAUR, PTGS2, PTX3, SELENOK, TGIF1, TREM1, TSC22D2, ZBTB43, ZFP36L2 and ZNF503; and
indicating the subject is eligible for treatment with a STING antagonist based on the expression level of the one or more genes determined in the determining step.

9. The method of claim 8 wherein levels of expression higher than a reference value indicate that the subject is eligible for treatment with a STING antagonist, whereas levels of expression lower than the reference value indicate that the subject is not eligible for treatment with a STING antagonist.

10. The method according claim 1 wherein the sample is a sample of monocytes.

11. The method of claim 10 wherein the sample further comprises dendritic cells.

12. The method according to claim 1 wherein the expression level of the one or more gene(s) is determined by RNA sequencing.

13. (canceled)

14. A method for diagnosis, comprising:
a) determining an—expression level of at least one gene selected from the group consisting of AC253572.2, ADRB2, ANKRD28, ANXA1, AQP9, AZIN1-AS1, IL1B, JARID2, KLF4, MIR4435-2HG, OTUD1, PLAUR, PTGS2, PTX3, SELENOK, TGIF1, TREM1, TSC22D2, ZBTB43, ZFP36L2 and ZNF503,
b) implementing an algorithm on data comprising or consisting of the expression levels determined at step a), as to obtain an algorithm output, the implementing step being computer-implemented; and
c) determining with the computer-implemented algorithm whether:
the subject suffers from a SAVI,
the subject achieves a response to treatment and/or
the subject is eligible for a treatment with a STING antagonist.

15. The method according to claim 5 wherein the sample is a sample of monocytes.

16. The method according to claim 15 wherein the sample further comprises dendritic cells.

17. The method according to claim 8 wherein the sample is a sample of monocytes.

18. The method according to claim 17 wherein the sample further comprises dendritic cells.

19. The method according to claim 14 wherein the sample is a sample of monocytes.

20. The method according to claim 19 wherein the sample further comprises dendritic cells.
